SEQUENCE LISTING (4 sequences)

SEQ ID NO: 1 - DNA, 21 bp, synthetic construct
  source 1..21: mol_type = other DNA
gctgcatgtggataaagccgt

SEQ ID NO: 2 - DNA, 20 bp, synthetic construct
  source 1..20: mol_type = other DNA
ttcggagtggagcagctgag

SEQ ID NO: 3 - DNA, 22 bp, synthetic construct
  source 1..22: mol_type = other DNA
tcccatcaccatcttccaggag

SEQ ID NO: 4 - DNA, 21 bp, synthetic construct
  source 1..21: mol_type = other DNA
agccttctccatggtggtgaa

(19) United States
(12) Patent Application Publication
(10) Pub. No.: US 2026/0306440 A1
(43) Pub. Date: Oct. 8, 2026

(54) USE OF SMALL MOLECULES TO INCREASE HYPOXIA INDUCIBLE FACTOR (HIF) ACTIVITY

(71) Applicant: BROWN UNIVERSITY, Providence, RI (US)

(72) Inventors: Wafik EL-DEIRY, Providence, RI (US); Shuai ZHAO, Providence, RI (US)

(21) Appl. No.: 19/164,441

(22) PCT Filed: Mar. 14, 2024
(86) PCT No.: PCT/US2024/019955

§ 371 Date: Sep. 11, 2025

Related U.S. Application Data
(60) Provisional application No. 63/452,344, filed on Mar. 15, 2023.

(51) Int. Cl. A61K 31/341; A61K 31/4025; A61K 31/4155

(52) U.S. Cl. CPC A61K 31/341; A61K 31/4025; A61K 31/4155

(57) ABSTRACT

A method of treating heart failure and/or a stroke in a subject in need thereof is provided. The method includes administering a therapeutically effective amount of a compound that upregulates HIF-1α transcriptional activity.

### CROSS REFERENCE TO RELATED APPLICATIONS

This application claims the benefit of U.S. Provisional Application No. 63/452,344 filed Mar. 15, 2023, which is incorporated herein by reference in its entirety.

### SEQUENCE LISTING

This application contains a sequence listing having the filename 0312021-01064_Sequence_Listing.xml, which is 5 KB in size, and was created on Mar. 1, 2024. The entire content of this sequence listing is incorporated herein by reference.

### FIELD

The present disclosure relates to therapeutics for the treatment of wounds, heart failure and strokes.

### BACKGROUND

Hypoxia-inducible factors (HIFs) are transcription factors that respond to decreases in available oxygen in the cellular environment, or hypoxia. When oxygen demand exceeds supply, the oxygen sensing pathway centred on the hypoxia inducible factor (HIF) is switched on and promotes adaptation to hypoxia by up-regulating genes involved in angiogenesis, erythropoiesis and glycolysis. The regulation of HIF is modulated through intricate regulatory mechanisms. Its protein stability is controlled by the oxygen sensing prolyl hydroxylase domain (PHD) enzymes and its transcriptional activity is controlled by the asparaginyl hydroxylase FIH (factor inhibiting HIF-1). HIF-1α acts as an oxygen stability transcription regulator and a main stimulant of adaptive response via upregulation of several target genes including genes involved in erythropoiesis, angiogenesis, glucose transport, and metabolism.

### SUMMARY

Various illnesses lead to chronic anemia including chronic kidney disease due to erythropoietin deficiency. Other illnesses include myelodysplastic syndrome. Hypoxia-inducible factor upregulates VEGF which helps healing wounds. Therapeutics that upregulate HIF could also have applications in heart failure and stroke. Small molecules that upregulate HIF1-alpha transcriptional activity may be used to treat these conditions/illnesses/diseases

Provided herein are compounds that upregulate HIF activity and/or increase the expression of HIFs, including HIF-1α.

Also provided are methods of treating a disease selected from heart failure and/or stroke, wounds, or chronic anemia, in a subject in need thereof, the method comprising administering a therapeutically effective amount of a compound that upregulates HIF-1α transcriptional activity, or a pharmaceutically acceptable salt thereof, to the subject.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1. Colorectal cancer cell line SW480 Normoxia 6h; expression of HIF-1α, Ran proteins in the presence of varying concentrations of PYR 41, C 646, 4E1 RCat, and Heclin.

FIG. 2. Colorectal cancer cell line SW480 Hypoxia 6h; expression of HIF-1α, Ran proteins in the presence of varying concentrations of PYR 41 and C 646.

FIG. 3. Renal cell carcinoma cell line RCC4 Normoxia 6h; expression of HIF-1α, Ran proteins in the presence of varying concentrations of PYR 41 and C 646.

FIG. 4. Colorectal cancer cell line SW480 Normoxia 6h; expression of HIF-1α, Ran, HIF-2a, GLUT1, VEGF proteins in the presence of varying concentrations of PYR 41, C 646, 4E1RCat, and Heclin.

FIG. 5. Colorectal carcinoma cell line HCT116 Normoxia 6h; using VEGF-HRE::dLUC reporter assay, and varying concentrations of PYR 41, C 646, 4E1RCat, and Heclin.

FIG. 6. Colorectal carcinoma cell line HCT116 Hypoxia 6h; using VEGF-HRE::dLUC reporter assay and varying concentrations of PYR 41, C 646, 4E1RCat, and Heclin.

FIG. 7. Colorectal carcinoma cell line HCT116 Normoxia 6h; using PGK1-HRE::dLUC varying concentrations of PYR 41, C 646, 4E1 RCat, and Heclin.

FIG. 8. Colorectal carcinoma cell line HCT116 Hypoxia 6h; using PGK1-HRE::dLUC reporter assay and varying concentrations of PYR 41, C 646, 4E1RCat, and Heclin.

FIG. 9. Colorectal carcinoma cell line HCT116 Normoxia 6h; using VEGF-HRE reporter assay and varying concentrations of 4E1 RCat.

FIG. 10. Colorectal carcinoma cell line HCT116 Normoxia 6h; using PGK1-HRE reporter assay and varying concentrations of 4E1RCat.

FIG. 11. Colorectal carcinoma cell line HCT116 Hypoxia 6h; using VEGF-HRE reporter assay and varying concentrations of 4E1RCat.

FIG. 12. HCT116 Hypoxia 6h; using PGK1-HRE reporter assay and varying concentrations of 4E1RCat.

FIG. 13. Colorectal cancer cell line SW480 Normoxia 6h; using VEGF-HRE reporter assay and varying concentrations of 4E1RCat.

FIG. 14. Colorectal cancer cell line SW480 Normoxia 6h; using PGK1-HRE reporter assay and varying concentrations of 4E1RCat.

FIG. 15. Colorectal cancer cell line SW480 Hypoxia 6h; using VEGF-HRE reporter assay and varying concentrations of 4E1RCat.

FIG. 16. Colorectal cancer cell line SW480 Hypoxia 6h; using PGK1-HRE reporter assay and varying concentrations of 4E1RCat.

FIG. 17. Colorectal carcinoma cell line HCT116 Normoxia 6h; using VEGF-HRE::dLUC reporter assay and varying concentrations of E1RCat.

FIG. 18. Colorectal carcinoma cell line HCT116 Normoxia 6h; using PGK1-HRE::dLUC reporter assay and varying concentrations of E1RCat.

FIG. 19. Colorectal carcinoma cell line HCT116 Hypoxia 6h; using VEGF-HRE::dLUC reporter assay and varying concentrations of E1RCat.

FIG. 20. Colorectal carcinoma cell line HCT116 Hypoxia 6h; using PGK1-HRE::dLUC reporter assay and varying concentrations of E1RCat.

FIG. 21. Colorectal cancer cell line SW480 Normoxia 6h; using VEGF-HRE::dLUC reporter assay and varying concentrations of E1RCat.

FIG. 22. Colorectal cancer cell line SW480 Normoxia 6h; using PGK1-HRE::dLUC reporter assay and varying concentrations of E1RCat.

FIG. 23. Colorectal cancer cell line SW480 Hypoxia 6h; using VEGF-HRE::dLUC reporter assay and varying concentrations of E1RCat.

FIG. 24. Colorectal cancer cell line SW480 Hypoxia 6h; using PGK1-HRE::dLUC reporter assay and varying concentrations of E1RCat.

FIG. 25 Colorectal carcinoma cell line HCT116 Normoxia 6h; using VEGF-HRE::dLUC reporter assay; cells were incubated with two different eIF4E siRNAs for 48 hours and treated with varying concentrations of E1RCat.

FIG. 26 Colorectal carcinoma cell line HCT116 Hypoxia 6h; using VEGF-HRE::dLUC reporter assay; cells were incubated with two different eIF4E siRNAs for 48 hours and treated with varying concentrations of E1RCat.

FIG. 27 Colorectal cancer cell line SW480 Normoxia 6h; using VEGF-HRE::dLUC reporter assay; cells were incubated with two different eIF4E siRNAs for 48 hours and treated with varying concentrations of E1RCat.

FIG. 28 Colorectal cancer cell line SW480 Hypoxia 6h; using VEGF-HRE::dLUC reporter assay; cells were incubated with two different eIF4E siRNAs for 48 hours and treated with varying concentrations of E1RCat.

FIG. 29 4E1RCat increases HIF-1α expression in colorectal carcinoma cancer cells HCT116 under normoxia. Cells were treated with 4E1 RCat at indicated doses for 6, 8 or 10 hours.

FIG. 30 shows expression of HIF-1α, and Ran proteins in the presence of varying concentrations of 4E1RCat in HT29 (colorectal cancer) and PANC1 (pancreatic cancer) cells; Normoxia 6h.

FIG. 31 shows that 4E1RCat increases erythropoietin (EPO) mRNA expression in colorectal carcinoma cancer cells HCT116. Cells were treated for indicated durations in normoxia or hypoxia. Extracted RNA was analyzed by real-time PCR. M is Carbobenzoxy-1-leucyl-1-leucyl-1-leucinal (MG132) (5 μM) a control. Statistical analysis: two-way anova+Dunnett's multiple comparisons test.

FIG. 32 shows that PYR-41 increases HIF-1α expression in colorectal carcinoma cancer cells HCT116 under hypoxia, in colorectal cancer cells SW480 under normoxia and hypoxia, and in renal cell carcinoma cells RCC4 under normoxia. N: normoxia; H: hypoxia.

### DETAILED DESCRIPTION

HIF-1α is the alpha subunit of HIF-1, and is a transcription factor that modulates the expression of a diverse group of genes that contribute to oxygen regulation. Under normoxic conditions, HIF-1α is hydroxylated and subsequently degraded. Knockdown of Smurf2 increases HIF-1α expression in normoxia.

Described herein are studies in which smurf2 (SMAD ubiquitination regulatory factor 2) inhibition and/or HIF-1α expression was studied. Three analogues of heclin were tested in reporter assays. The structures of heclin and the three analogues are shown below:

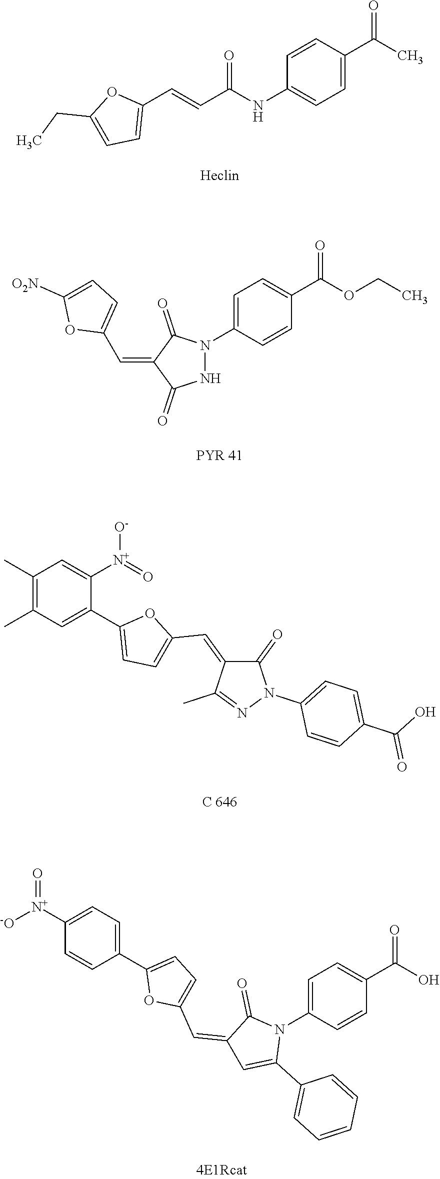

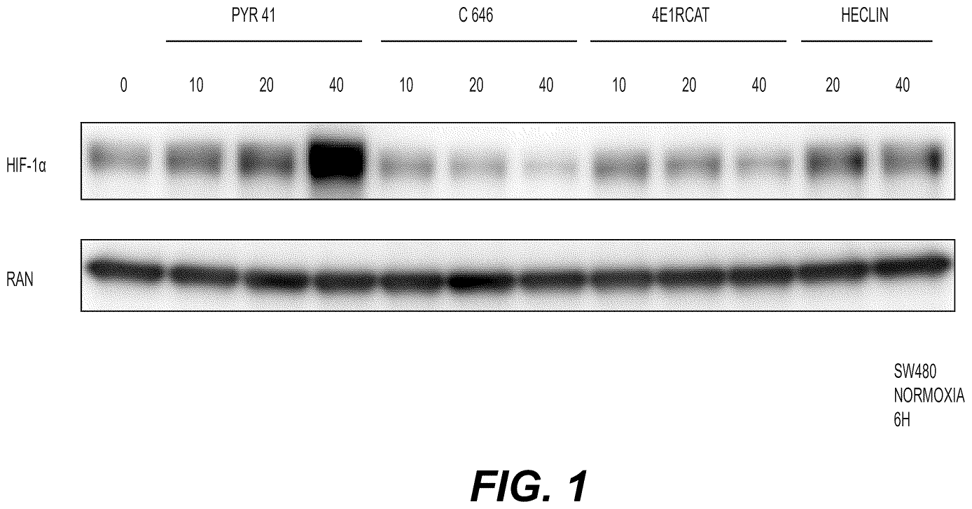

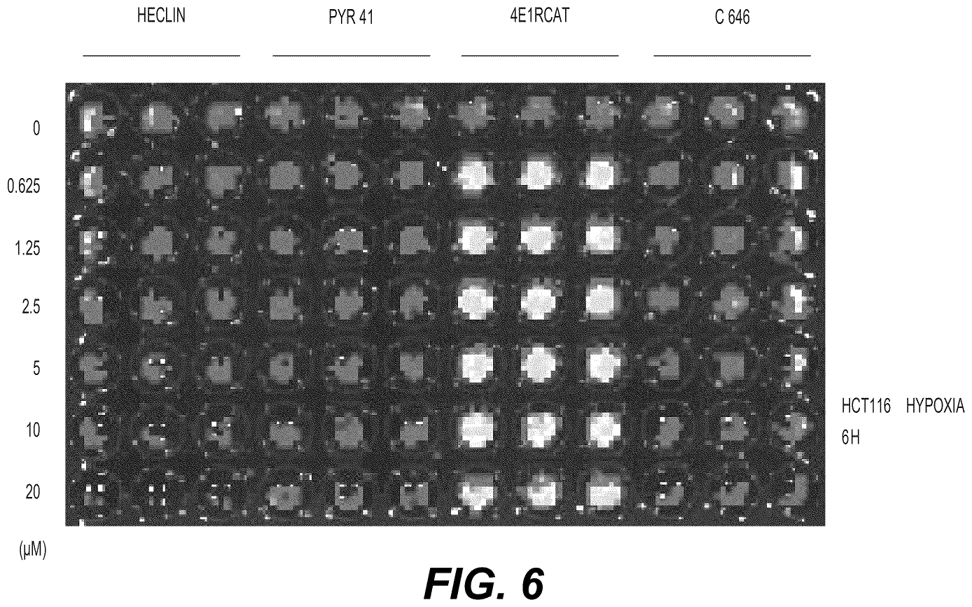

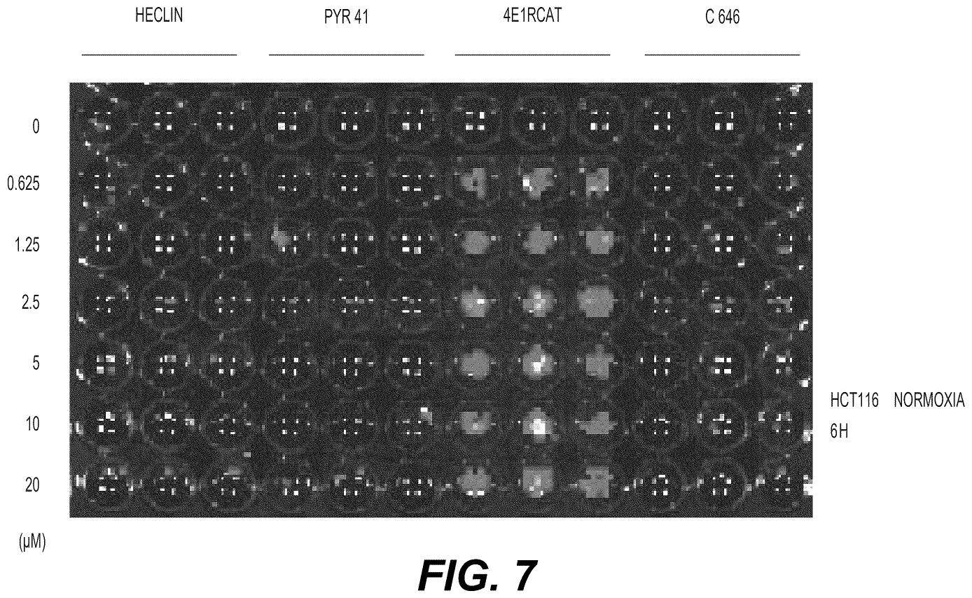

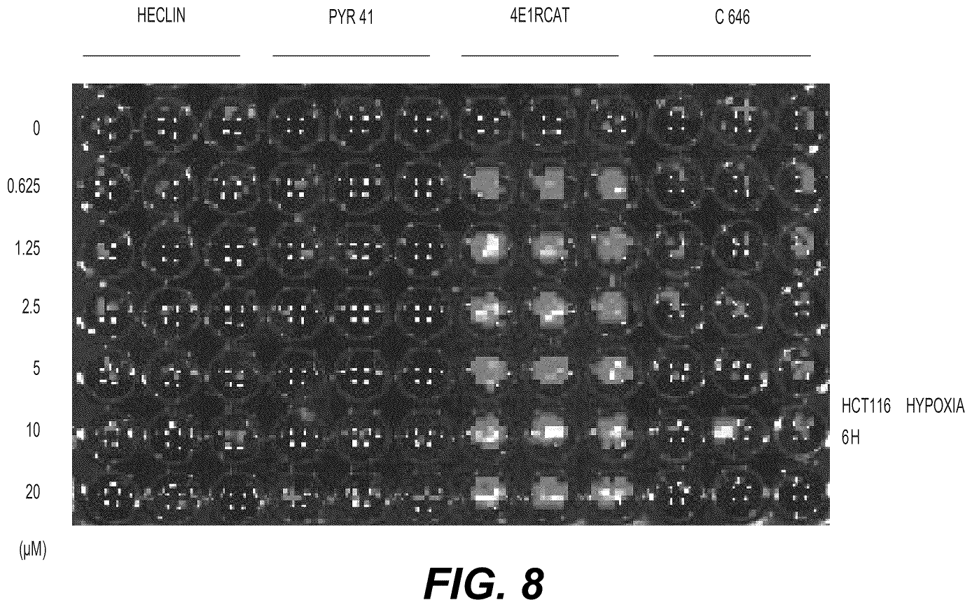

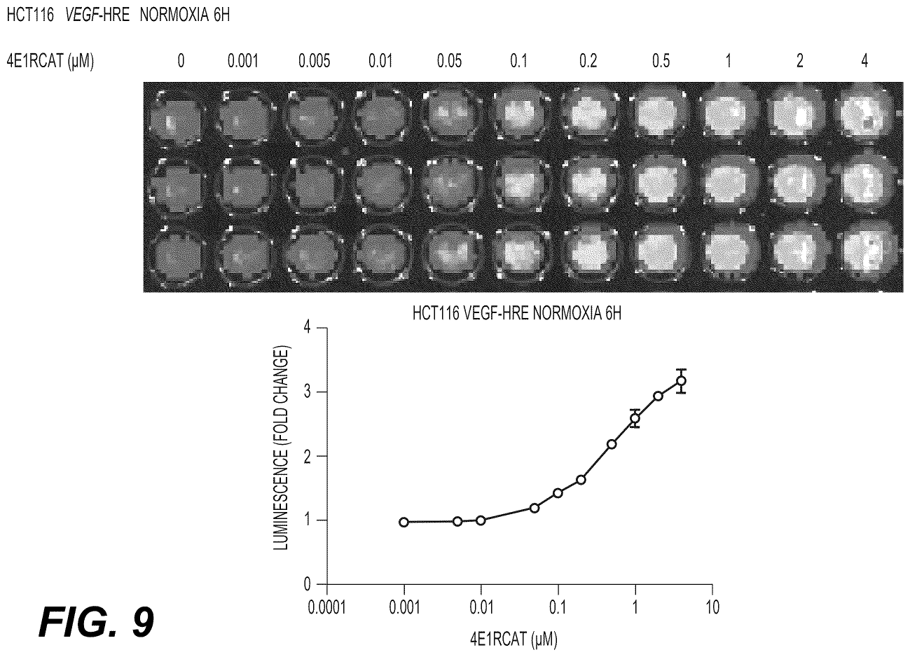

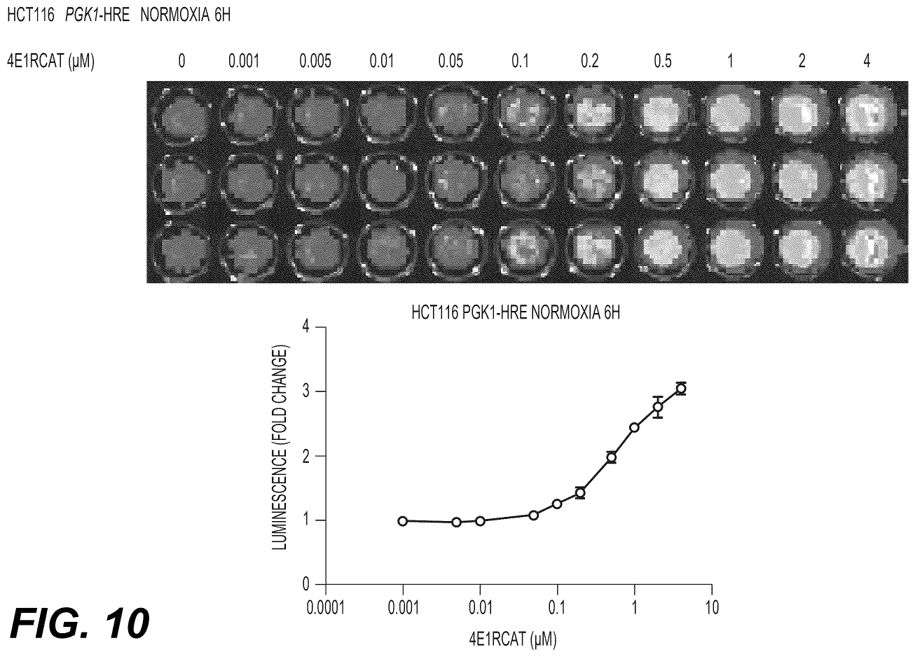

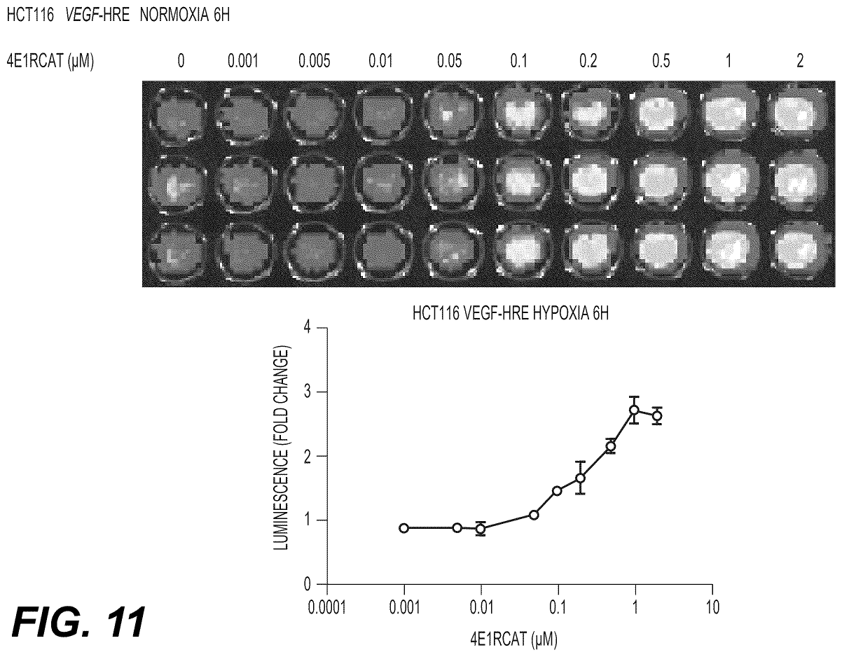

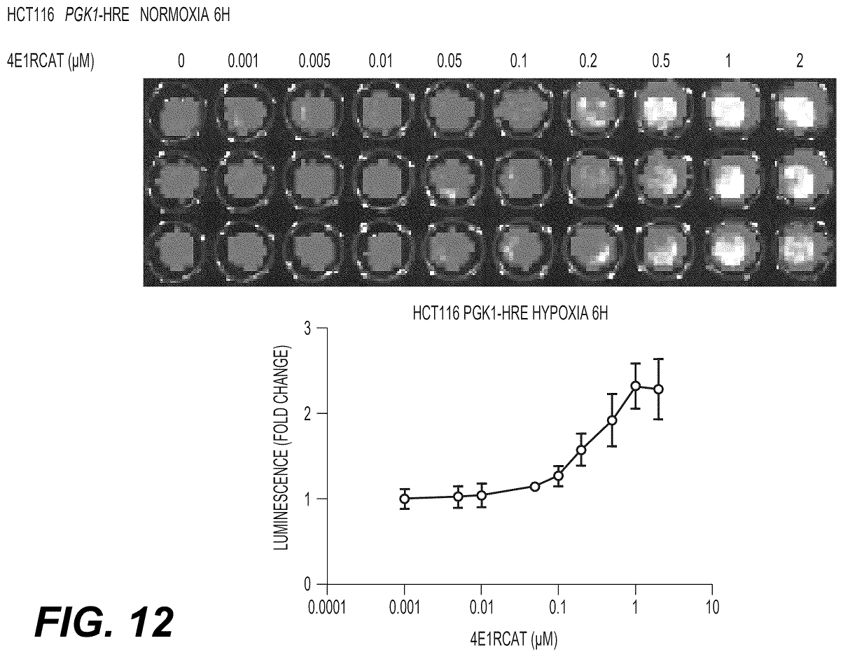

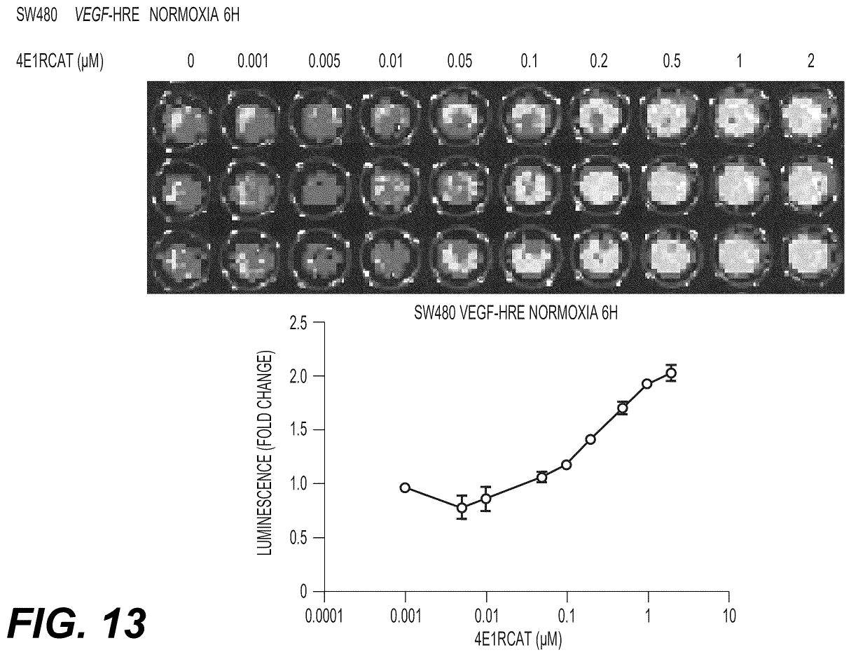

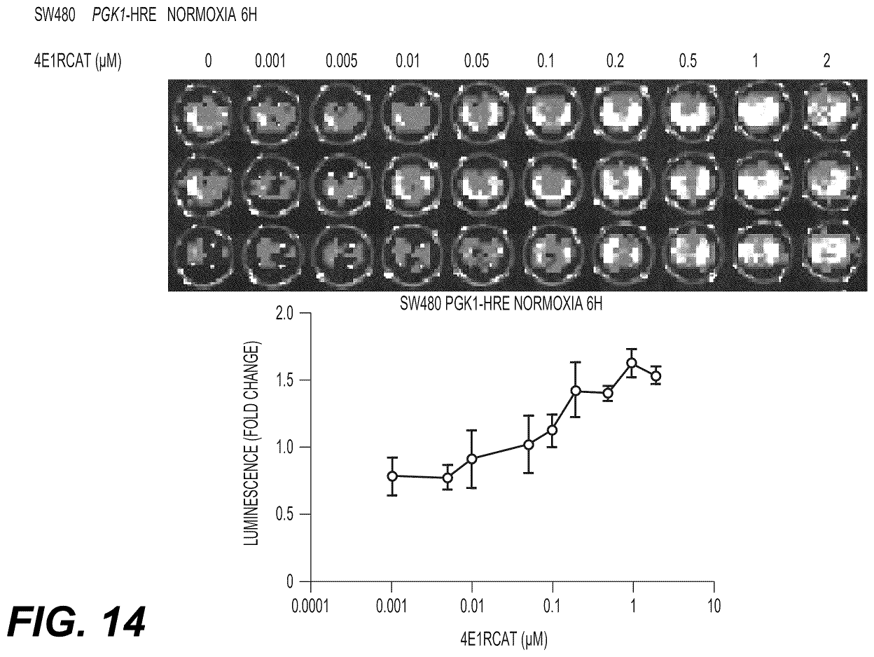

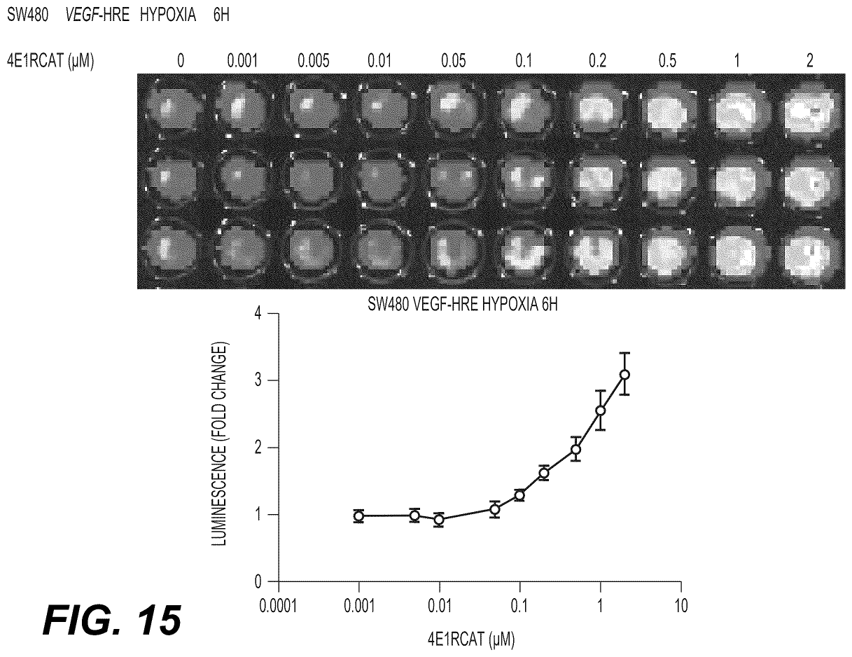

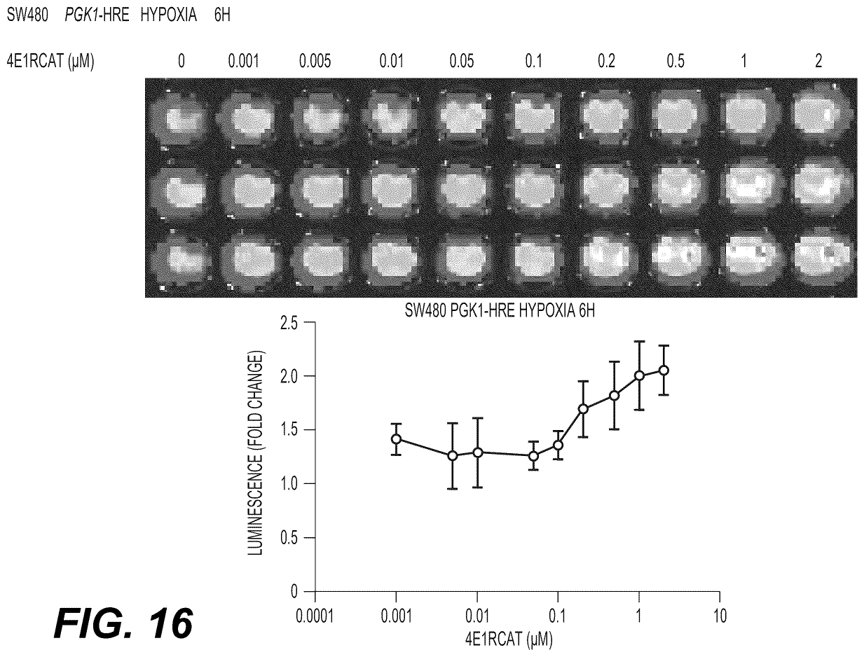

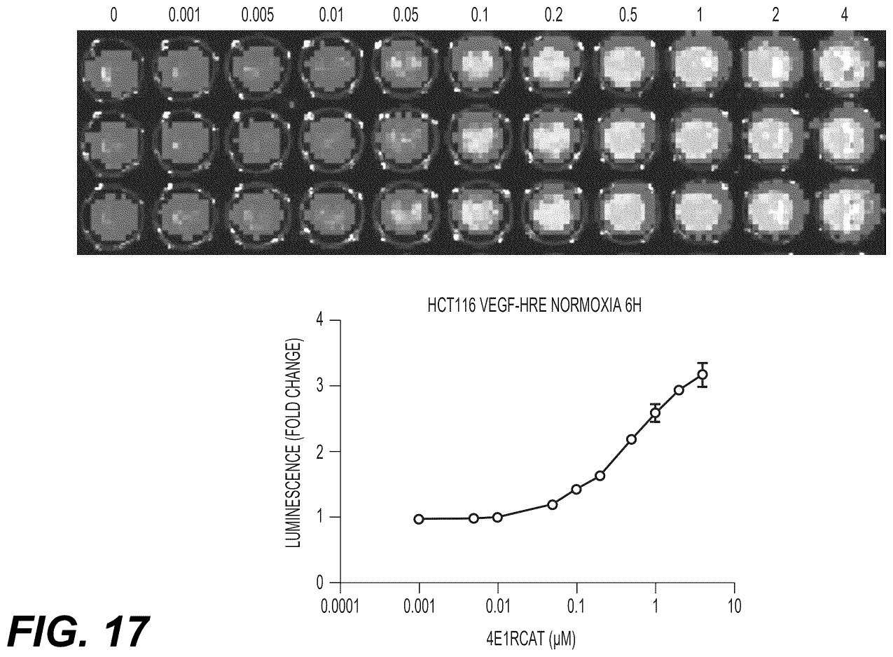

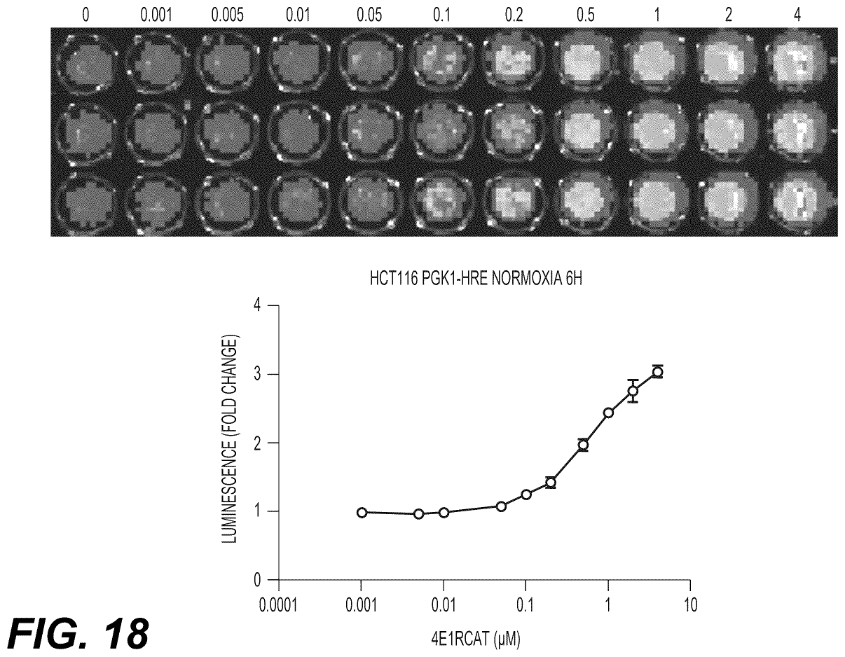

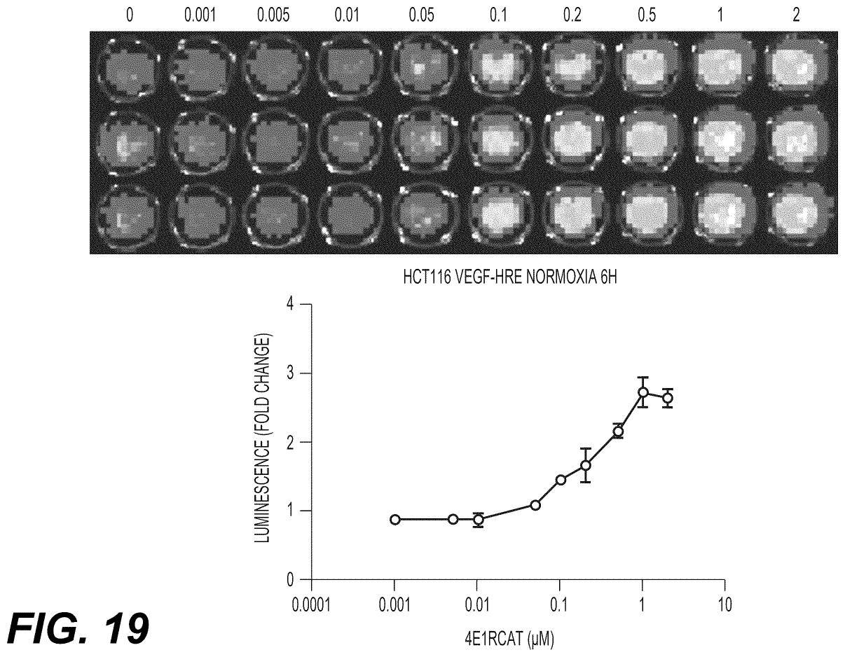

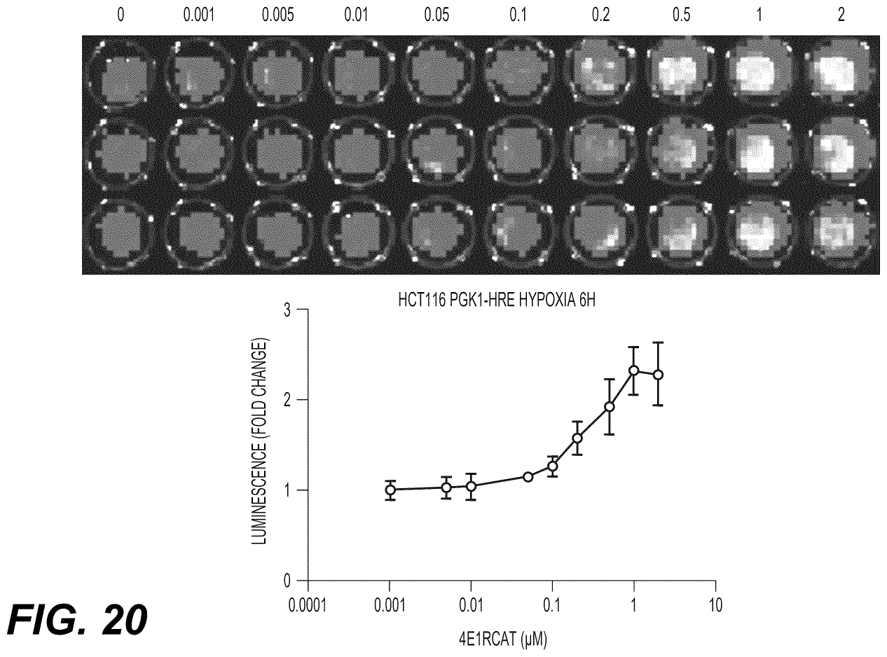

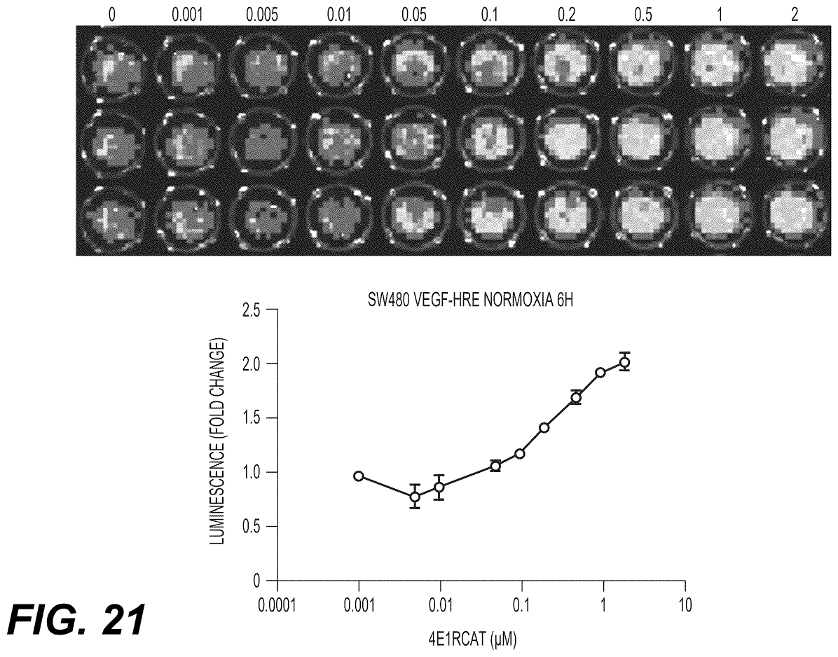

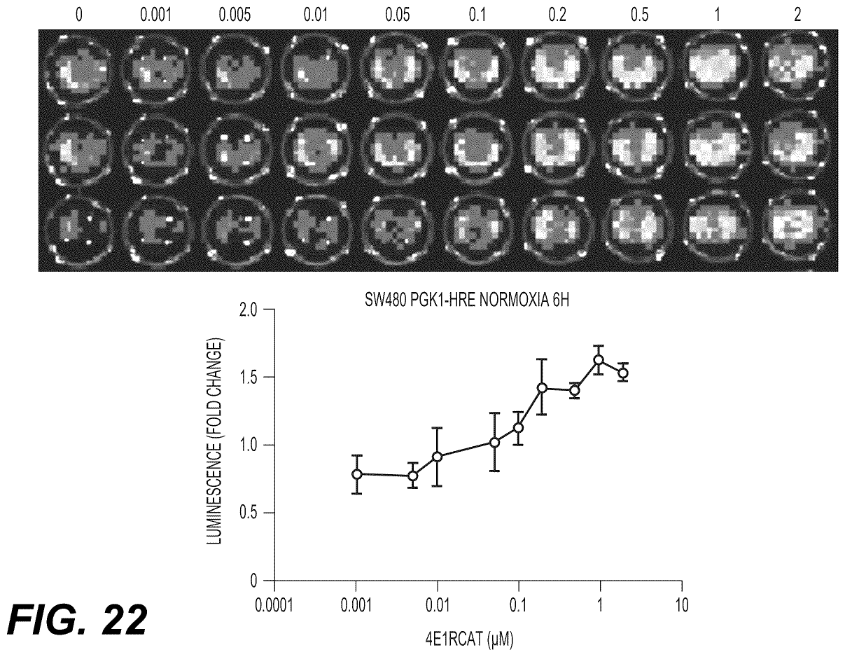

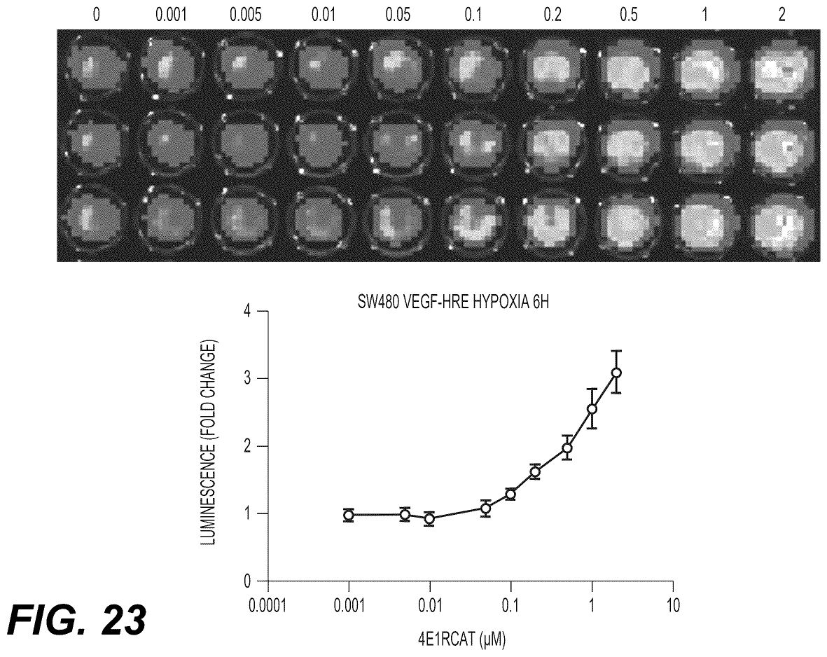

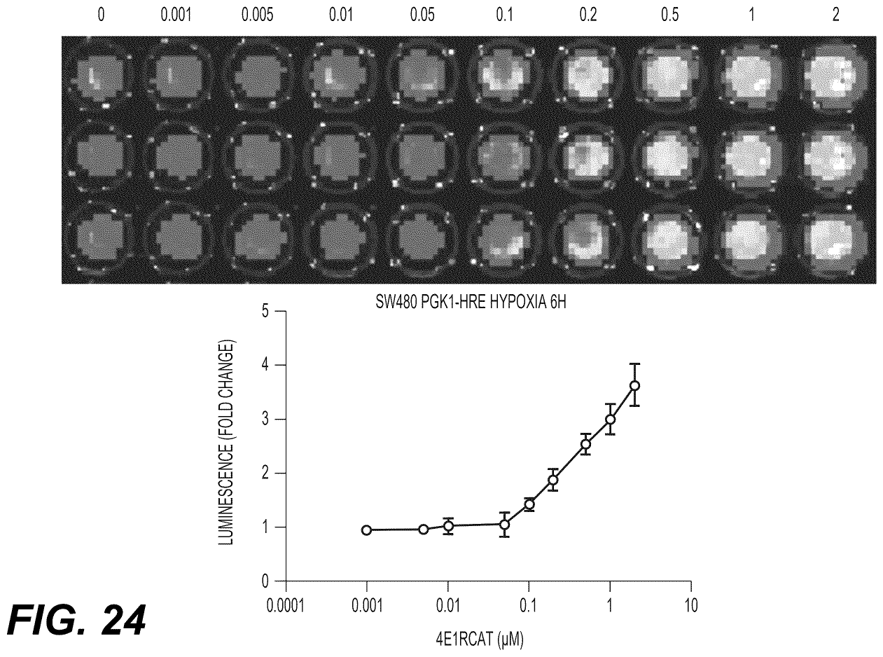

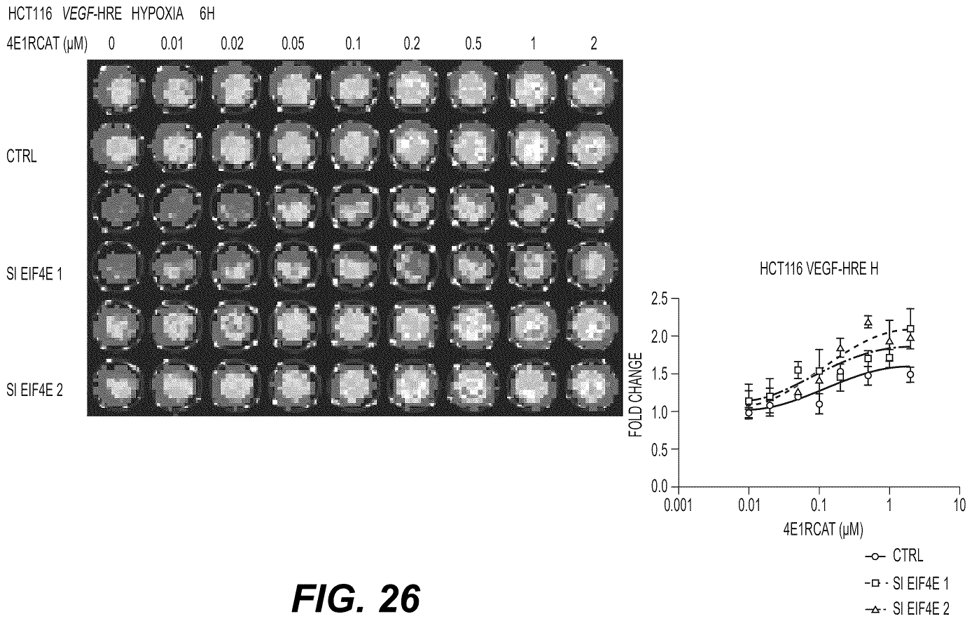

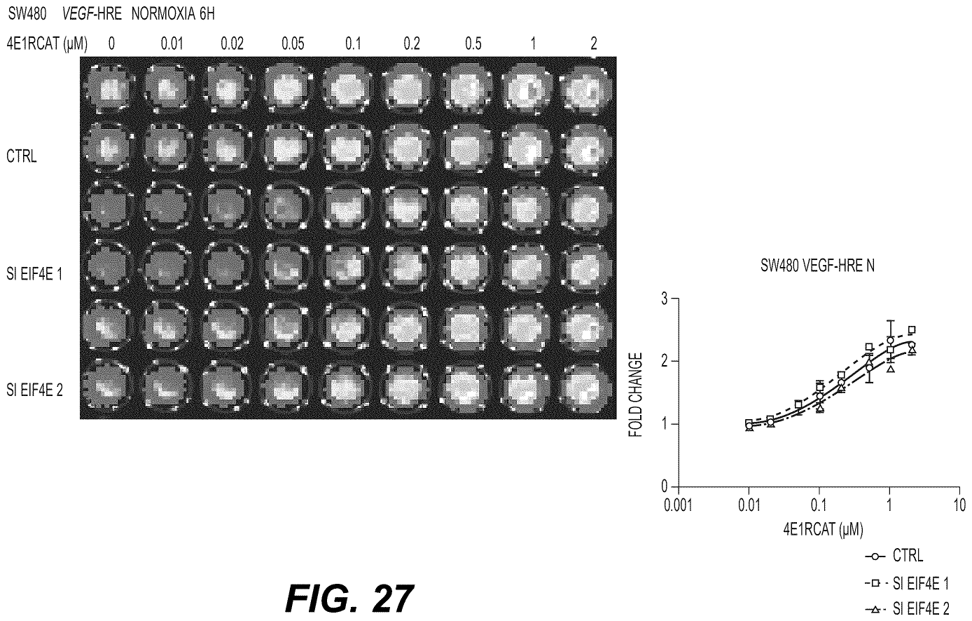

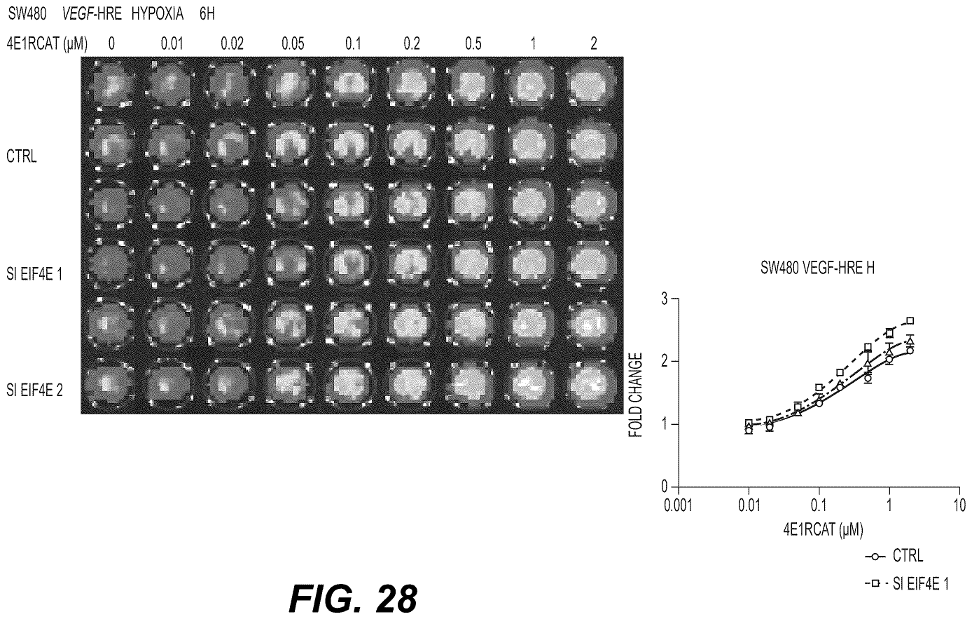

Cell lines with different HIF target reporters (VEGF, PGK1) were generated. Results of the foregoing studies are shown in FIGS. 1-32.

While overexpression of HIF-1α is observed in a number of cancers and predicts unfavorable prognosis, there are other diseases where upregulation of HIF-1α can be beneficial. In some embodiments, upregulation of HIF-1α transcriptional activity may be achieved by inhibition of Smurf2.

HIF-1α is known to upregulate VEGF. In some embodiments, upregulation of HIF-1a may upregulate VEGF and treat diseases associated with VEGF (e.g., angiogenesis plays a role in wound healing and VEGF regulates angiogenesis).

Aspects of the present disclosure are directed to methods of treating heart failure and/or a stroke in a subject in need thereof. The methods include administering a therapeutically effective amount of a compound that upregulates HIF-1α transcriptional activity. Another aspect of the present disclosure is directed to methods of treating wounds in a subject in need thereof. The methods include administering a therapeutically effective amount of a compound that upregulates HIF-1α transcriptional activity.

In some embodiments, the compound is a pharmaceutically acceptable salt thereof.

In some embodiments, a pharmaceutical composition includes the compound or pharmaceutically acceptable salt thereof.

In some embodiments, provided herein is a method of treating a disease selected from heart failure and/or stroke, wounds, or chronic anemia, in a subject in need thereof, the method comprising administering a therapeutically effective amount of a compound that upregulates HIF-1α transcriptional activity, or a pharmaceutically acceptable salt thereof, to the subject.

In some embodiments, the compound that upregulates HIF-1 transcriptional activity is a compound of formula

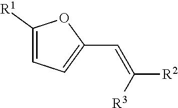

wherein
R¹ is NO₂, C₁₋₃ alkyl, or aryl substituted with 1-4 groups selected from NO₂, or C₁₋₃ alkyl;
R³ is H and R² is —C(═O)—NH-aryl, wherein aryl is optionally substituted with —COOH, —C(═O)C₁₋₃ alkyl, or —C(═O)O—C₁₋₃ alkyl,
or R² and R³ together with the atom to which they are attached from a 5-membered heterocycle comprising 1-2 nitrogen atoms and optionally substituted with 1-3 substituents selected from oxo, C₁₋₃ alkyl, or aryl optionally substituted with —COOH, —C(═O)C₁₋₃ alkyl, or —C(═O)O—C₁₋₃ alkyl.

In some embodiments, the compound that upregulates HIF-1α transcriptional activity is selected from

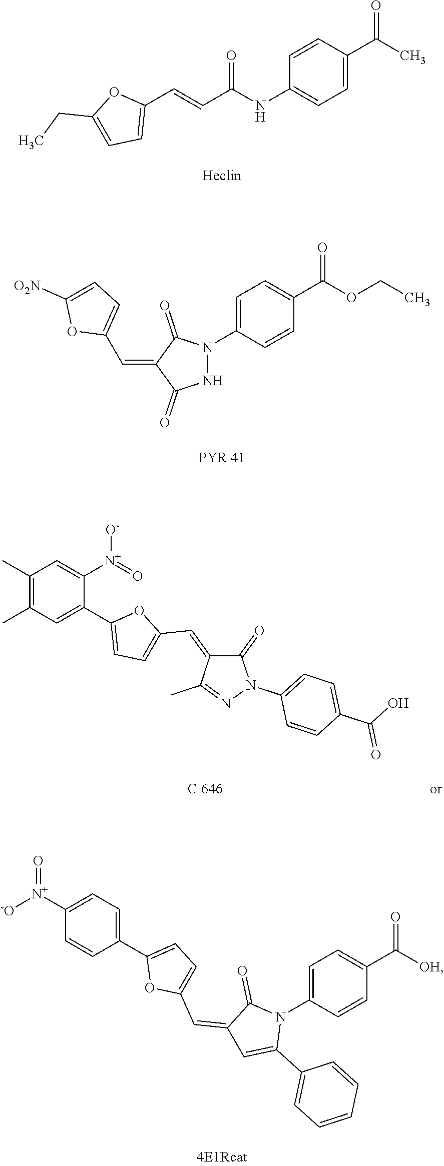

or a pharmaceutically acceptable salt thereof.

In some embodiments, the disease is heart failure and/or a stroke. In some embodiments, the heart failure is congestive heart failure, systolic failure, or diastolic failure. In some embodiments, a stroke is an ischemic stroke, a hemorrhagic stroke, or a transient ischemic attack.

In some embodiments, the disease is a wound. In some embodiments a wound is a laceration, an abrasion, a pressure ulcer, a surgical wound, a puncture, a burn, an avulsion, or any other wound that may benefit from upregulation of HIF-1α activity.

In some embodiments, the disease is chronic anemia. In some embodiments, the chronic anemia is associated with cancer (e.g., myelodysplastic syndromes), chronic kidney disease, ulcerative colitis, arthritis, diabetes, or any other underlying disease that may cause anemia.

In some embodiments, the compound is administered orally, or parenterally. In some embodiments, when the disease is a wound, the compound may be administered topically (e.g., as a dermal patch).

In some embodiments, the compound is Heclin. In some embodiments, the compound is PYR 41. In some embodiments, the compound is C 646. In some embodiments, the compound is 4E1Rcat.

In some embodiments, the upregulation of HIF-1α transcriptional activity occurs under normoxia. In some embodiments, the upregulation of HIF-1α transcriptional activity occurs under hypoxia.

In some embodiments, the subject is not suffering from cancer.

Further provided herein is a method of upregulating transcriptional activity of erythropoietin (EPO) in a subject in need thereof comprising administering a therapeutically effective amount of a compound of formula

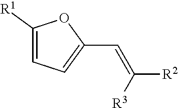

wherein
R¹ is NO₂, C₁₋₃ alkyl, or aryl substituted with 1-4 groups selected from NO₂, or C₁₋₃ alkyl;
R³ is H and R² is —C(═O)—NH-aryl, wherein aryl is optionally substituted with —COOH, —C(═O)C₁₋₃ alkyl, or —C(═O)O—C₁₋₃ alkyl,
or R² and R³ together with the atom to which they are attached from a 5-membered heterocycle comprising 1-2 nitrogen atoms and optionally substituted with 1-3 substituents selected from oxo, C₁₋₃ alkyl, or aryl optionally substituted with —COOH, —C(═O)C₁₋₃ alkyl, or —C(═O)O—C₁₋₃ alkyl,
or a pharmaceutically acceptable salt thereof, to the subject.

In some embodiments, the compound is selected from Heclin, PYR 41, C 646, or 4E1Rcat, or a pharmaceutically acceptable salt thereof. In some embodiments, the subject is suffering from chronic anemia.

In some embodiments, the upregulation of EPO transcriptional activity occurs under normoxia. In some embodiments, the upregulation of EPO transcriptional activity occurs under hypoxia.

Further provided herein is a method of upregulating VEGF by upregulating HIF-1α in a subject in need thereof comprising administering a therapeutically effective amount of a compound selected from Heclin, PYR 41, C 646, or 4E1Rcat, or a pharmaceutically acceptable salt thereof, to the subject. In some embodiments, the subject has a wound and upregulation of VEGF by upregulating HIF-1α allows for angiogenesis and/or wound healing.

Also provided herein is a pharmaceutical composition comprising a compound that upregulates HIF-1α transcriptional activity, or a pharmaceutically acceptable salt thereof, and a pharmaceutically acceptable carrier.

In some embodiments of the composition, the compound is selected from Heclin, PYR 41, C 646, or 4E1Rcat, or a pharmaceutically acceptable salt thereof

In some embodiments, the composition is an oral or parenteral composition. In some embodiments, the composition is a topical composition.

The present disclosure also provides pharmaceutical compositions that include effective amounts of the compounds, and the pharmaceutically acceptable salts thereof described above, and a pharmaceutically acceptable carrier. In certain embodiments, the disclosure also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Definitions

“Alkyl” means a saturated alkyl chain having 1 to 6 carbon atoms which may be straight chained or branched. Examples thereof include methyl, ethyl, propyl, isopropyl, n-butyl, isobutyl, tert-butyl, n-pentyl, isopentyl, neopentyl and hexyl.

“Aryl” means phenyl or naphthyl.

“Heterocycle” means a saturated or partially unsaturated cycle comprising 1-2 nitrogen atoms. Examples of heterocycle include and are not limited to

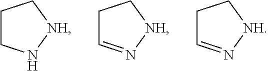 [SMILES: C1CNNC1 ; C1=NNCC1 ; C1=NNCC1]

The optional substituents on a heterocycle may on a carbon atom of the heterocycle or a nitrogen atom of a heterocycle, or both.

“Oxo” refers to C═O.

As used herein, the term “optionally substituted” may be used interchangeably with “unsubstituted or substituted”. The term “substituted” refers to the replacement of one or more hydrogen atoms in a specified group with a specified radical. It will be appreciated by persons of skill in the art that, because oxo is a divalent radical, there are circumstances in which it will not be appropriate as a substituent (e.g. on phenyl). In one embodiment, 1, 2, or 3 hydrogen atoms are replaced with a specified radical.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

The terms “effective amount” or “therapeutically effective amount” refer to an amount, i.e. a dosage, of therapeutic agent administered to a subject (e.g., a mammalian subject, i.e. a human subject), either as a single dose or as part of a series of doses, which is effective to produce a desired therapeutic effect (e.g., effective for influencing, reducing or inhibiting the activity of or preventing activation of a kinase, or effective at bringing about a desired in vivo effect in an animal, preferably, a human, such as reduction in intraocular pressure).

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

“Normoxia” refers to an environment (e.g., in an incubator, or in vivo in a tissue sample) having an oxygenation of about 21% by volume.

“Hypoxia” refers to an environment (e.g., in an incubator, or in vivo in a tissue sample) having an oxygenation range of about 0.1% to about 5% by volume. In some embodiments, hypoxia refers to an environment (e.g., in an incubator, or in vivo in a tissue sample) having an oxygenation of about 0.5% by volume.

As used herein, “pharmaceutically acceptable salts” refers to an ionizable therapeutic agent that has been combined with a counter-ion to form a neutral complex. Lists of suitable salts are found, for example, in Remington's Pharmaceutical Sciences, 17th ed., Mack Publishing Company, Easton, Pa., 1985, p. 1418 and Journal of Pharmaceutical Science, 66, 2 (1977).

The terms “pharmaceutical” and “pharmaceutically acceptable” may refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier” means a carrier that is useful for the preparation of a pharmaceutical composition that is: generally compatible with the other ingredients of the composition, not deleterious to the recipient, and neither biologically nor otherwise undesirable. “A pharmaceutically acceptable carrier” includes both one and more than one carrier. Embodiments include carriers for topical, ocular, parenteral, intravenous, intraperitoneal intramuscular, sublingual, nasal, and oral administration. “Pharmaceutically acceptable carrier” also includes agents for preparation of aqueous dispersions and sterile powders for injection or dispersions.

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

The term “treatment” may refer to the application of one or more specific procedures used for the amelioration of a disease. In certain embodiments, the specific procedure is the administration of one or more pharmaceutical agents. “Treatment” of an individual (e.g. a mammal, such as a human) or a cell is any type of intervention used in an attempt to alter the natural course of the individual or cell. Treatment includes, but is not limited to, administration of a therapeutic agent or a pharmaceutical composition, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset.

### Routes of Administration and Dosage Forms

The pharmaceutical compositions of the present disclosure include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein can be administered orally. Compositions of the present disclosure suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (e.g., glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

The pharmaceutical compositions of the present disclosure may be administered in the form of suppositories for rectal administration. These compositions can be prepared by mixing a compound of the present disclosure with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present disclosure is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

Examples of useful dermatological compositions which can be used to deliver the compounds and therapeutic agents to the skin are known in the art.

The compounds and therapeutic agents of the present disclosure may be incorporated into compositions for coating an implantable medical device, such as prostheses, artificial valves, vascular grafts, stents, or catheters. Suitable coatings and the general preparation of coated implantable devices are known in the art. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present disclosure provides an implantable drug release device impregnated with or containing a compound or a therapeutic agent, or a composition comprising a compound of the present disclosure or a therapeutic agent, such that said compound or therapeutic agent is released from said device and is therapeutically active.

### Dosages and Regimens

In the pharmaceutical compositions of the present disclosure, a compound is present in an effective amount (e.g., a therapeutically effective amount).

Effective doses/amounts may vary, depending on the diseases treated, the severity of the disease, the route of administration, the sex, age and general health condition of the subject, excipient usage, and the possibility of co-usage with other therapeutic treatments such as use of other agents and the judgment of the treating physician.

In some embodiments, an effective amount of the compounds, nucleic acids and the pharmaceutically acceptable salts thereof described above, can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of the compounds, nucleic acids and the pharmaceutically acceptable salts thereof described above is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

In some embodiments, provided herein are packaged dosage forms, comprising a container holding a therapeutically effective amount of the compounds, nucleic acids and the pharmaceutically acceptable salts thereof described above, and instructions for using the dosage form in accordance with one or more of the methods provided herein.

The present dosage forms and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (for example, Irgacure 184, 2959), preferably water-soluble initiators (for example, Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (for example, by the addition of specific product information leaflets) into suitable containers (boxes, etc.).

According to further embodiments, the described dosage forms can also be provided in kit form combined with other components necessary for administration of the material to the patient. For example, disclosed kits, such as for use in the treatments described herein, can further comprise, for example, administration materials.

The kits may be designed in various forms based on the specific deficiencies they are designed to treat.

The dosage forms provided herein may be prepared and placed in a container for storage at ambient or elevated temperature. This is beneficial because transportation of commercially viable dosage forms may benefit from stability at temperatures greater than those requiring refrigeration or sub-freezing environments during transportation and storage at the site of use.

When the dosage forms provided herein are stored in a polyolefin plastic container as compared to, for example, a polyvinyl chloride plastic container, discoloration of the dosage form may be reduced. Without wishing to be bound by theory, the container may reduce exposure of the container's contents to electromagnetic radiation, whether visible light (for example, having a wavelength of about 380-780 nm) or ultraviolet (UV) light (for example, having a wavelength of about 190-320 nm (UV B light) or about 320-380 nm (UV A light)). Some containers also include the capacity to reduce adherence or adsorption of the active ingredient to the surface of the container, which could effectively dilute the concentration of active ingredient in the contained solution. Some containers also include the capacity to reduce exposure of the container's contents to infrared light, or a second component with such a capacity. Some containers further include the capacity to reduce the exposure of the container's contents to heat or humidity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper container, cardboard container, paperboard container, metallic film container, or foil container, or a combination thereof, to further reduce exposure of the container's contents to UV, visible, or infrared light. Articles of manufacture benefiting from reduced discoloration, decomposition, or both during storage, include dosage forms that include compounds, nucleic acids and the pharmaceutically acceptable salts thereof. The dosage forms provided herein may need storage lasting up to, or longer than, three months; in some cases up to, or longer than one year. The containers may be in any form suitable to contain the contents—for example, a bag, a bottle, or a box.

### EXAMPLES

### Example 1: Stable Cell Line Establishment

HCT116 or SW480 cells were transfected using lipofectamine 2000 (Invitrogen, USA) with pGL4.22-VEGF-HRE::dLUC or pGL4.22-PGK1-HRE::dLUC plasmids expressing the luciferase reporter with hypoxia response element (HRE) derived from the human PGK1 promoter or the human VEGF promoter (Walton Z. E. et al., Cell. 2018 Jun. 28; 174(1):72-87). Cells were cultured and selected in 2 μg/mL (for HCT116) or 4 μg/mL (for SW480) puromycin-containing DMEM medium (Cytiva HyClone) after 24 h of transfection. Individual clones were picked and expanded. Verification of the luciferase reporter-bearing cells were performed by measuring the bioluminescence in hypoxia upon addition of the substrate (luciferin) using the IVIS imaging system.

### Example 2: Luciferase Reporter Assay

Luciferase-reporter expressing cells were seeded in 96-well black plates at a density of 2~4×10⁴ cells/well. Treatment with compounds were performed on the next day for 6 h under normoxia or hypoxia. After substrate (luciferin) addition, plates were imaged for luminescence intensity in the IVIS imaging system.

### Example 3: qRT-PCR Methods and Primers

Total RNA was extracted from cells using the RNeasy Mini Kit (Qiagen). cDNA was obtained by reverse transcription with random primers using the SuperScript II First-Strand Synthesis System (Invitrogen). Real-time PCR reactions were performed with SYBR Green Master Mix (Applied Biosystems) using the QuantStudio™ 6 Pro Real-Time PCR System.

EPO forward sequence:
(SEQ ID NO: 1)
5′-GCT GCA TGT GGA TAA AGC CGT-3′
EPO reverse sequence:
(SEQ ID NO: 2)
5′-TTC GGA GTG GAG CAG CTG AG-3′
GAPDH forward sequence:
(SEQ ID NO: 3)
5′-TCC CAT CAC CAT CTT CCA GGA G-3′
GAPDH reverse sequence:
(SEQ ID NO: 4)
5′-AGC CTT CTC CAT GGT GGT GAA-3′

### Example 4: Hypoxic Culture

Hypoxic conditions (0.5% O₂) were achieved by culturing cells in a hypoxia chamber (HypOxygen H35 HypOxystation) which controls and maintains a stable level of oxygen, carbon dioxide, temperature and humidity.

Normoxia refers to a regular cell culture condition at 21% oxygen concentration.

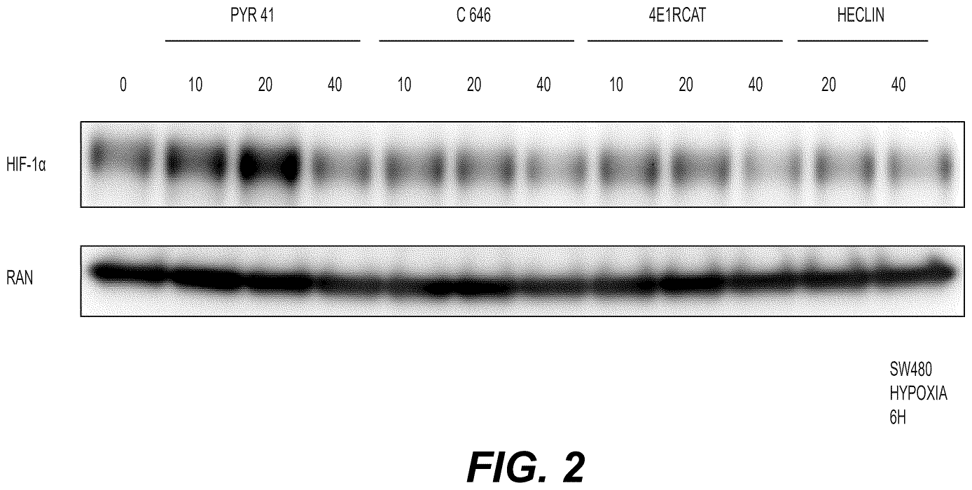

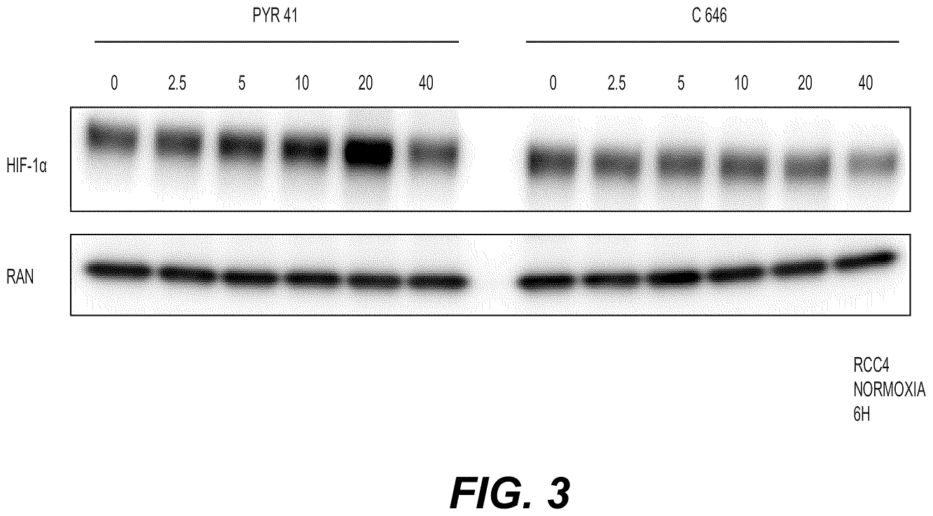

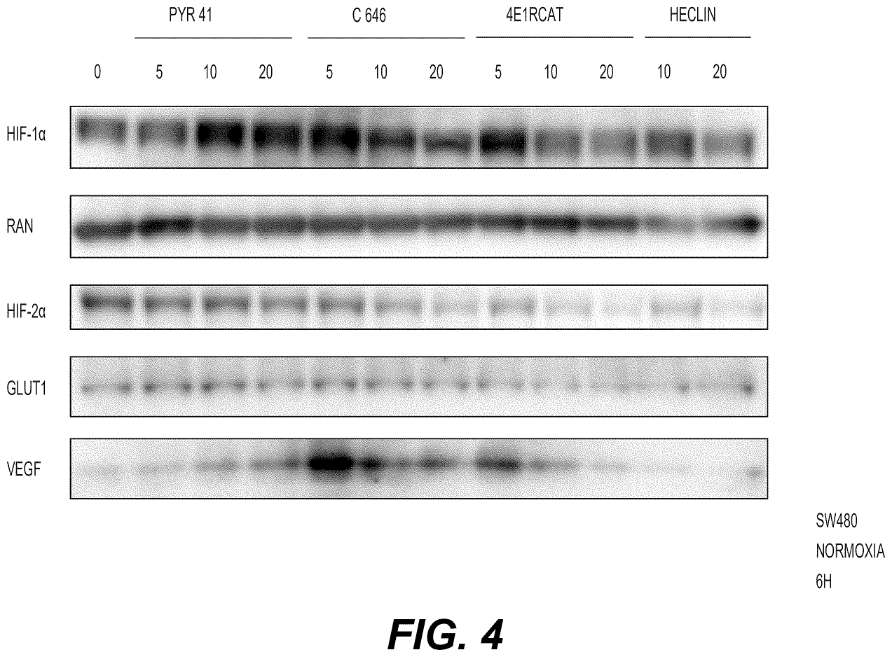

The data shown in FIG. 1, FIG. 2, FIG. 3 and FIG. 4 demonstrates that compounds Heclin, PYR 41, C 646, and 4E1Rcat increase expression of HIF-1α under normoxia and under hypoxia. The data shown in FIG. 4 further demonstrates that compounds Heclin, PYR 41, C 646, and 4E1Rcat increase expression VEGF expression under normoxia.

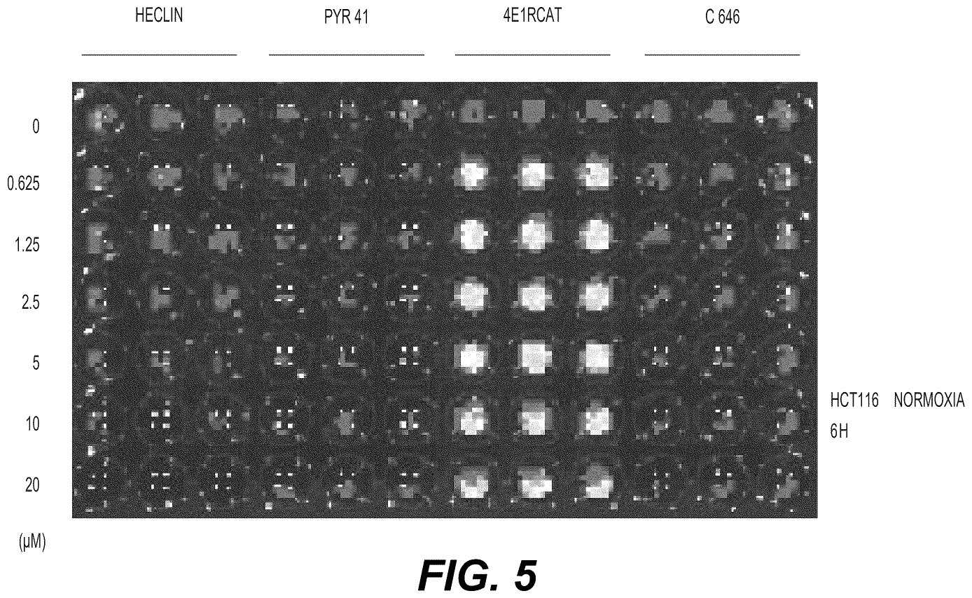

The data shown in figures, i.e., FIG. 5 to FIG. 24, shows that 4E1Rcat increases expression of VEGF and PGK1 as measured in a luciferase assay.

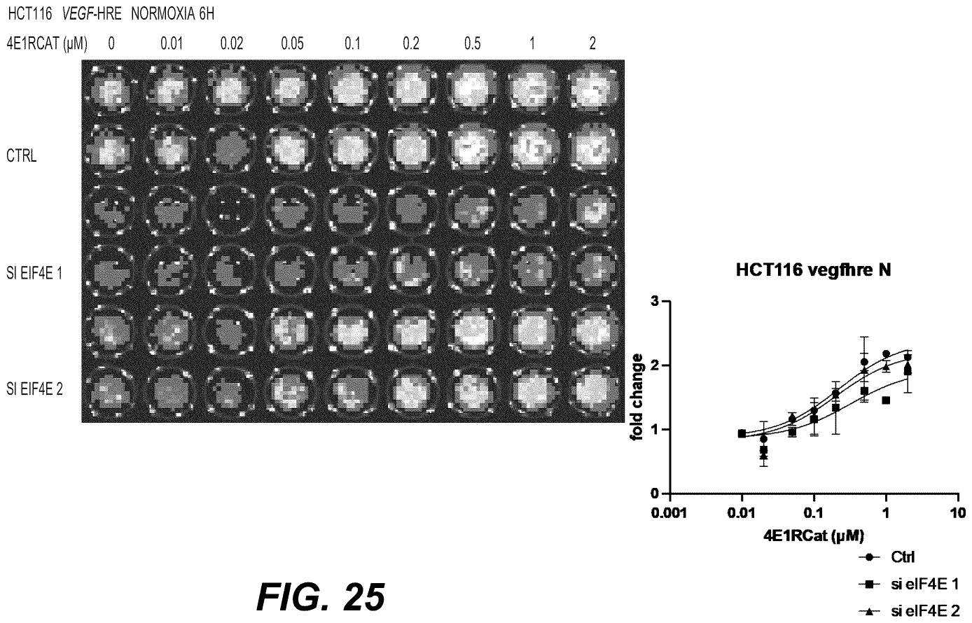

The data shown in figures, i.e., FIG. 25 to FIG. 28 demonstrates that despite incubation with siRNA EIF4E, after exposure to compound eE1Rcat, VEGF is upregulated in HCT116 and SW480 cells under normoxia and hyoxia as measured by a luciferase assay.

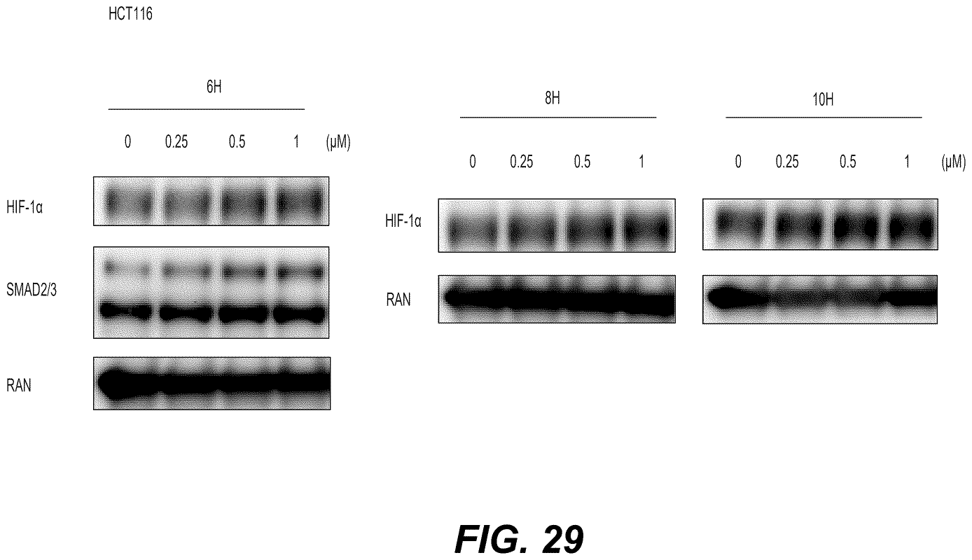

FIG. 29 demonstrates that 4E1RCat increases HIF-1α expression in colorectal carcinoma cancer cells HCT116 under normoxia. Cells were treated with 4E1RCat at indicated doses for 6, 8 or 10 hours.

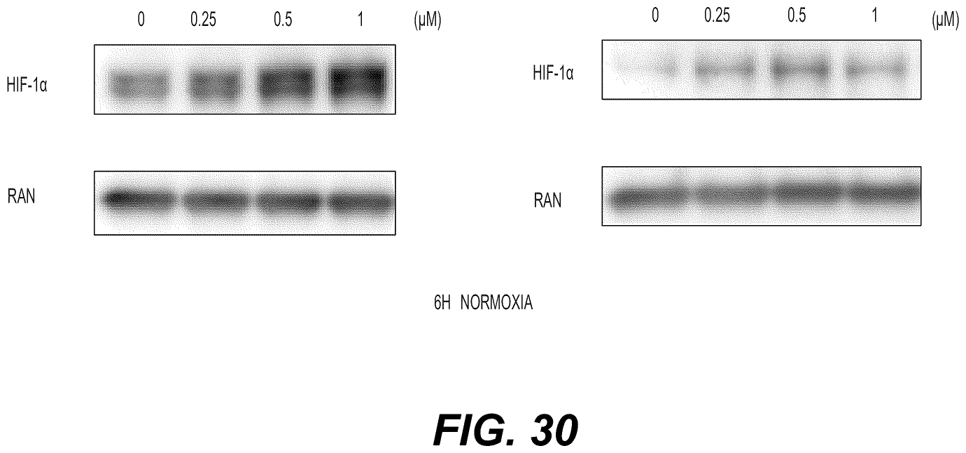

FIG. 30 demonstrates that expression of HIF-1α increases in the presence of varying concentrations of 4E1RCat in HT29 (colorectal cancer) and PANC1 (pancreatic cancer) cells; Normoxia 6h.

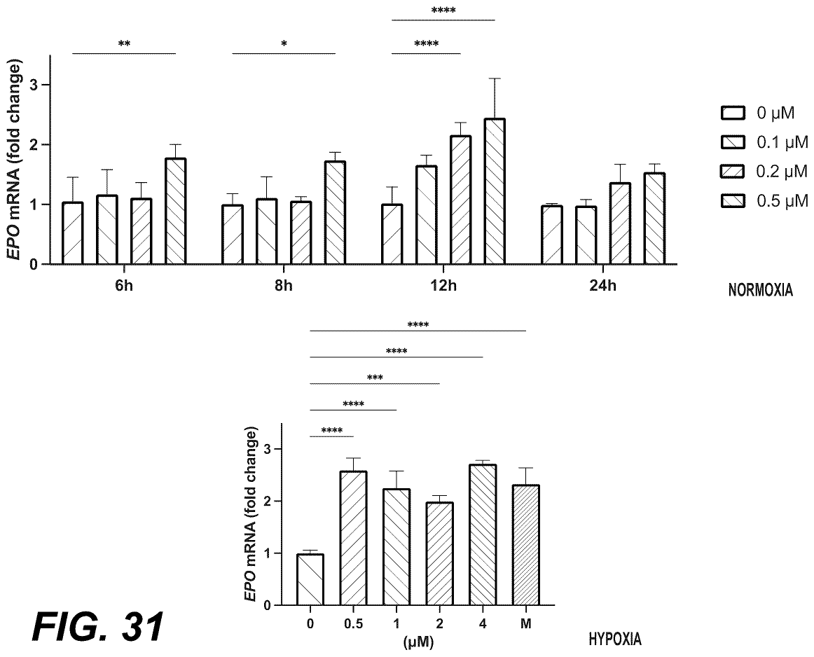

FIG. 31 demonstrates that 4E1RCat increases erythropoietin (EPO) mRNA expression in colorectal carcinoma cancer cells HCT116. Cells were treated for indicated durations in normoxia or hypoxia. Extracted RNA was analyzed by real-time PCR. M: MG132 (5 μM). Statistic analysis: two-way anova+Dunnett's multiple comparisons test.

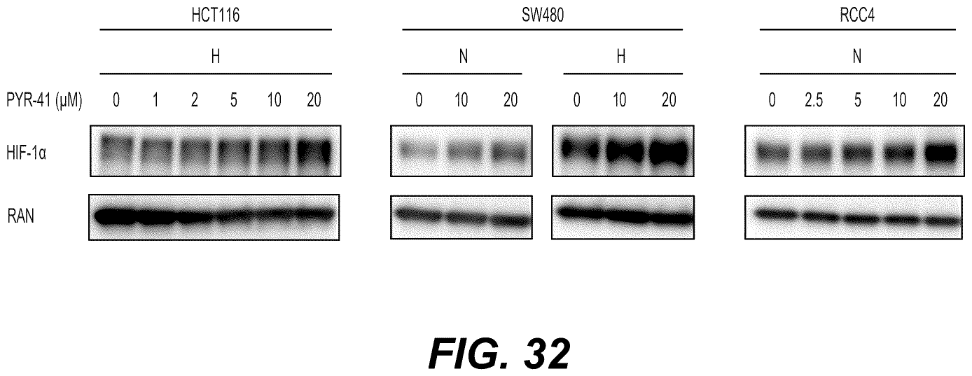

FIG. 32 demonstrates that PYR-41 increases HIF-1α expression in colorectal carcinoma cancer cells HCT116 under hypoxia, in colorectal cancer cells SW480 under normoxia and hypoxia, and in renal cell carcinoma cells RCC4 under normoxia. N: normoxia; H: hypoxia.

Accordingly, the compounds Heclin, PYR 41, C 646, and 4E1Rcat can be used to upregulate HIF-1α and/or VEGF and/or erythropoietin (EPO) under normoxia and/or hypoxia.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

Furthermore, references to patents and printed publications may have been made in this specification. Each of the above-cited references and printed publications are individually incorporated herein by reference in their entirety.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

1. A method of treating a disease selected from heart failure and/or stroke, wounds, or chronic anemia, in a subject in need thereof, the method comprising administering a therapeutically effective amount of a compound that upregulates HIF-1α transcriptional activity, or a pharmaceutically acceptable salt thereof, to the subject.

2. The method of claim 1, wherein the compound that upregulates HIF-1α transcriptional activity is a compound of formula 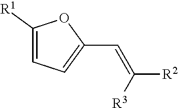
wherein
R¹ is NO₂, C₁₋₃ alkyl, or aryl substituted with 1-4 groups selected from NO₂, or C₁₋₃ alkyl;
R³ is H and R² is —C(═O)—NH-aryl, wherein aryl is optionally substituted with —COOH, —C(═O)C₁₋₃ alkyl, or —C(═O)O—C₁₋₃ alkyl,
or R² and R³ together with the atom to which they are attached from a 5-membered heterocycle comprising 1-2 nitrogen atoms and optionally substituted with 1-3 substituents selected from oxo, C₁₋₃ alkyl, or aryl optionally substituted with —COOH, —C(═O)C₁₋₃ alkyl, or —C(═O)O—C₁₋₃ alkyl,
or a pharmaceutically acceptable salt thereof.

3. The method of claim 1, wherein the compound that upregulates HIF-1α transcriptional activity is selected from 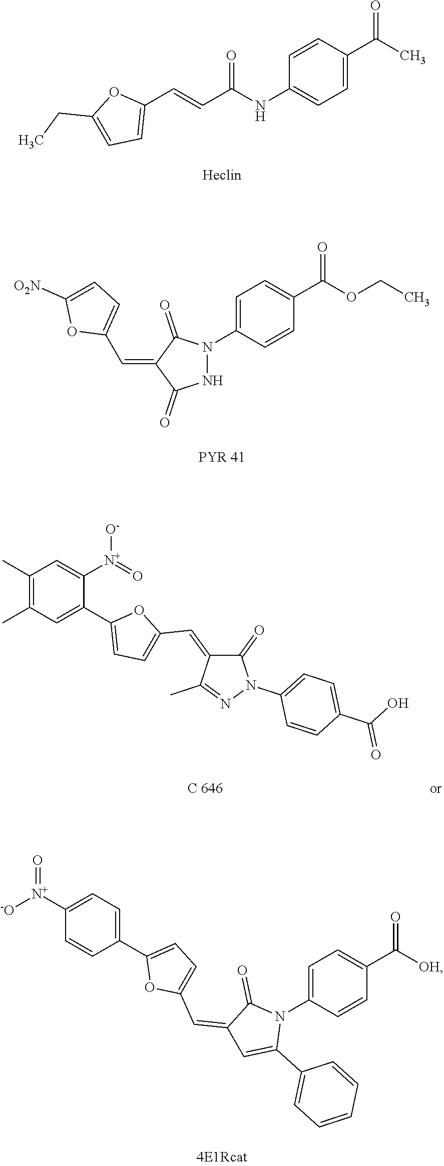
or a pharmaceutically acceptable salt thereof.

4. The method of claim 1, wherein the disease is heart failure and/or a stroke.

5. The method of claim 1, wherein the disease is a wound.

6. The method of claim 1, wherein the disease is chronic anemia.

7. The method of claim 1, wherein the compound is administered orally, or parenterally.

8. The method of claim 1, wherein the compound is Heclin.

9. The method of claim 1, wherein the compound is PYR 41.

10. The method of claim 1, wherein the compound is C 646.

11. The method of claim 1, wherein the compound is 4E1Rcat.

12. The method of claim 1, wherein the upregulation of HIF-1α transcriptional activity occurs under normoxia.

13. The method of claim 1, wherein the upregulation of HIF-1α transcriptional activity occurs under hypoxia.

14. The method of claim 1, wherein the subject is not suffering from cancer.

15. A method of upregulating transcriptional activity of erythropoietin (EPO) in a subject in need thereof comprising administering a therapeutically effective amount of a compound of formula 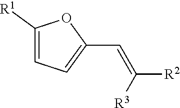
wherein
R¹ is NO₂, C₁₋₃ alkyl, or aryl substituted with 1-4 groups selected from NO₂, or C₁₋₃ alkyl;
R³ is H and R² is —C(═O)—NH-aryl, wherein aryl is optionally substituted with —COOH, —C(═O)C₁₋₃ alkyl, or —C(═O)O—C₁₋₃ alkyl,
or R² and R³ together with the atom to which they are attached from a 5-membered heterocycle comprising 1-2 nitrogen atoms and optionally substituted with 1-3 substituents selected from oxo, C₁₋₃ alkyl, or aryl optionally substituted with —COOH, —C(═O)C₁₋₃ alkyl, or —C(═O)O—C₁₋₃ alkyl,
or a pharmaceutically acceptable salt thereof, to the subject.

16. The method of claim 15, wherein the compound is selected from Heclin, PYR 41, C 646, or 4E1Rcat, or a pharmaceutically acceptable salt thereof.

17. The method of claim 15, wherein the subject suffers from chronic anemia.

18. The method of claim 15, wherein the upregulation of EPO transcriptional activity occurs under normoxia.

19. The method of claim 15, wherein the upregulation of EPO transcriptional activity occurs under hypoxia.

20. A pharmaceutical composition comprising a compound that upregulates HIF-1α transcriptional activity, or a pharmaceutically acceptable salt thereof, and a pharmaceutically acceptable carrier.

21. The pharmaceutical composition of claim 20, wherein the compound is selected from Heclin, PYR 41, C 646, or 4E1Rcat, or a pharmaceutically acceptable salt thereof

22. The pharmaceutical composition of claim 20, wherein the composition is an oral or parenteral composition.
